(19) United States
(12) Patent Application Publication
(10) Pub. No.: US 2026/0283653 A1
(43) Pub. Date: Sep. 24, 2026

(54) STRUT IMPLANT FOR SUPPORTING SPINAL IMPLANT CONSTRUCTS

(71) Applicant: Spinal Simplicity, LLC, Overland Park, KS (US)

(72) Inventors: Timothy L. Lasswell, Calgary (CA); Stewart D. McLachlin, Toronto (CA); Fred H. Nicholls, Calgary (CA)

(21) Appl. No.: 19/572,335

(22) Filed: Mar. 19, 2026

Related U.S. Application Data
(60) Provisional application No. 63/776,085, filed on Mar. 22, 2025.

(51) Int. Cl. A61B 17/70; A61B 17/00

(52) U.S. Cl. CPC A61B 17/7064; A61B 17/7007; A61B 17/7061; A61B 2017/00477

(57) ABSTRACT

An implant support system for providing support to spinal implants, such as pedicle screw-rod constructs having a rod connector connecting the screw to the rod. The implant support system may comprise a strut implant having a base and an extension extending from the base. The extension may form a support surface. The base may couple to the spinal implant at, for instance, the rod, the screw or the rod connector. The support surface may contact the screw of the spinal implant to provide contact support to the spinal implant to disperse a total stress applied to the screw and rod to improve spinal implant stability such that screw and rod deformity/breakage is avoided. The support surface may contact the cortical bone of a vertebra implanted with a screw to support the spinal implant and load applied to the screw and rod to improve spinal implant stability.

### RELATED APPLICATIONS

This non-provisional patent application claims priority benefit, with regard to all common subject matter, of U.S. Provisional Patent Application No. 63/776,085, filed Mar. 22, 2025, and entitled “PEDICLE SCREW STRUT SUPPORT DEVICE.” The above-identified application is hereby incorporated by reference in its entirety.

### BACKGROUND

### 1. Field

Embodiments of the current disclosure generally relate to support structures for providing and/or maintaining rigidity to a portion of a spinal implant. More specifically, embodiments of the current disclosure relate to systems and methods for providing additional stabilization to fixating devices, so the fixating devices do not deform or fail, such as providing additional stabilization to a portion of a spinal implant via a strut.

### 2. Related Art

Unstable burst fractures occurring in spines of patients may cause kyphotic deformities, height loss, and/or posterior ligamentous injury, thus necessitating elective and non-elective surgical treatment. Spinal segment fixation, using short or long segment constructs, enables spinal stabilization to prevent further deformity and patient harm, while permitting early weightbearing prior to boney fusion. Currently, use of short segment constructs spanning few spinal levels are prone to early mechanical failure prior to bony fusion, unless supplemented with anterior column stabilization. However, such supplemental anterior column stabilization is associated with increased morbidity and technical challenges. Alternatively, long segment fixation may be used, which involves the spanning of multiple uninjured levels to increase rigidity and reduce mechanical failure, at the expense of spinal mobility and patient comfort.

Most short segment fixation systems include pedicle screws, such as monoaxial or polyaxial screws. However, short segment fixation systems applying such screws have resulted in high failure rates, depending on the type of interconnection mechanisms and subassemblies used. For example, short segment constructs that use polyaxial screws are prone to failure from slipping at the screw shaft/tulip connector interface, leading to progressive kyphotic deformity and eventual screw or rod breakage due to high stresses at the base of the shaft or the rod/tulip interface. Similarly, short segment constructs using monoaxial screws or Schanz pins are prone to breaking first at the screw or rod, followed by a progressive kyphotic deformity. In both cases, the short segment constructs fail due to insufficient mechanical properties of the interconnection mechanisms (tulip/screw interfaces) or subassemblies (rod or screw shafts). Implant failure before fracture healing and/or bony union can result in poor patient outcomes such as ongoing pain and sagittal deformity.

To mitigate the failures of short segment fixation systems, long segment fixation is used to improve mechanical properties of the posterior spinal implant construct, but unfortunately involves instrumenting additional, uninjured levels of the spine. For example, to improve mechanical properties, long segments often span upwards of five vertebrae for lumbar burst factures and extend through the undamaged spine up to L4 for sacral fractures. Spanning multiple uninjured levels increases the invasiveness of the surgical procedure and forfeits spinal mobility for the patient. Thus, though long segment fixation may provide improved mechanical properties, there are disadvantages with such procedures, which is why short segments are still currently used, even given their decreased mechanical stability and durability.

Thus, what is needed is an implant construct and/or system that improves the mechanical strength and stability of posterior constructs with the goal of maintaining construct rigidity and stability, as well as spinal alignment, while minimizing the number of instrumented vertebral levels. For example, a short segment construct that prevents implant failure and minimizes the quantity of instrumented vertebral levels required for maintaining spinal alignment, fracture healing and/or bony fusion is desired. Additionally, a system is needed for enabling improved stabilization of the spine through mechanical improvements to pedicle screw-rod constructs.

While relevant to short segment fixation, a system is needed that enables the use of a supportive device with any fixating device, including long segment fixation, where mechanical properties of the spinal implant would further improve from additional structural support to the spinal implant. Such a system may combat forces that would otherwise deform or break implant constructs by dissipating and/or distributing the load and reducing stress concentrations.

### SUMMARY

Embodiments of the present disclosure relate to strut implants for providing improved rigidity and stability to a portion of a fixating devices such as those present within a spinal implant (e.g., pedicle screw/rod construct). The strut implants may be secured to a spinal implant such as a posterior pedicle screw-rod construct to improve mechanical properties by making the spinal implant more stable and distributing forces otherwise concentrated at the screw or screw/rod interface. The strut implant may provide load-bearing support by directly contacting the spinal implant proximate an interface corresponding with the mechanical failure of the spinal implant. Such mechanical failure may contribute to a spinal irregularity, such as spinal kyphosis. Additionally, the strut implant may enable structural support or stabilization of a vertebra by interfacing with the vertebra itself rather than contacting a surface of the spinal implant.

In some aspects, the techniques described herein relate to a strut implant. The strut implant may include a base configured to couple to a spinal implant. The strut implant may include a strut extension extending from the base. The strut extension may define a support surface. The support surface may be configured to support a screw of the spinal implant or cortical bone implanted with the spinal implant to provide structural support.

In some aspects, the techniques described herein relate to an implant support system including a spinal implant for providing spinal fixation. The spinal implant may include at least one screw configured to be inserted into a vertebra and at least one rod coupled to the at least one screw. Additionally, the system may further include at least one strut implant having a base that couples the at least one strut implant to the spinal implant. Furthermore, the at least one strut implant may comprise a strut extension extending from the base and defining a support surface. The support surface may be configured to contact the at least one screw to thereby provide structural support to the spinal implant.

In some aspects, the techniques described herein relate to an implant support system including a spinal implant. The spinal implant may include a pedicle screw configured to screw into a vertebra and a rod at which the pedicle screw may be coupled. Further, the system may include a strut implant including a base and a strut extension. The base may couple the strut implant to at least one of the pedicle screw or the rod. The strut extension may extend from the base such that the strut extension forms a support surface. The support surface may be curved and configured to abut a cortical bone of the vertebra when the pedicle screw is screwed into the vertebra. Moreover, the support surface may be configured to support the spinal implant to redistribute forces applied to the pedicle screw and the rod.

In some aspects, the techniques described herein relate to a method for stabilizing a spinal implant. Such method may include providing the spinal implant including at least one screw with a shaft and at least one rod. Additionally, the method may comprise providing at least one strut implant including a base and an extension extending from the base that forms a support surface. The method may further involve inserting the at least one screw into a vertebra, attaching the at least one screw to the at least one rod, placing the support surface against the shaft of the at least one screw, and attaching the base of the at least one strut implant to the at least one rod or the at least one screw.

This summary is provided to introduce a selection of concepts in a simplified form that are further described below in the detailed description. This summary is not intended to identify key features or essential features of the claimed subject matter, nor is it intended to limit the scope of the claimed subject matter. Other aspects and advantages of the present disclosure will be apparent from the following detailed description of the embodiments and the accompanying drawing figures.

### BRIEF DESCRIPTION OF THE DRAWING FIGURES

Embodiments of the present disclosure are described in detail below with reference to the attached drawing figures, wherein:

FIG. 1 depicts a perspective view of an exemplary implant support system for some embodiments;

FIG. 2A depicts a perspective view of an exemplary proximal interface strut for some embodiments;

FIG. 2B depicts a side view of an exemplary implant support system for some embodiments using the proximal interface strut implanted in the spine for some embodiments;

FIG. 2C depicts a perspective view of a second embodiment of a proximal interface strut for some embodiments;

FIG. 2D depicts a detailed view of a second embodiment of an implant support system using the second embodiment of the proximal interface strut implanted in the spine for some embodiments;

FIG. 2E depicts a perspective view of a third embodiment of a proximal interface strut for some embodiments;

FIG. 2F depicts a perspective view of a third embodiment of an implant support system using the third embodiment of the proximal interface strut implanted in the spine for some embodiments;

FIG. 2G depicts a perspective view of multiple implant support systems using the third embodiment of the proximal interface strut implanted in the spine some embodiments;

FIG. 3A depicts a perspective view of an exemplary distal interface strut for some embodiments;

FIG. 3B depicts a perspective view of an exemplary distal interface strut containing a lattice structure for some embodiments;

FIG. 3C depicts a perspective detailed view of an exemplary implant support system using the exemplary distal interface strut and implanted in the spine for some embodiments;

FIG. 3D depicts a perspective detailed view of multiple implant support systems using the distal interface strut and implanted in the spine for some embodiments;

FIG. 3E depicts a perspective detailed view of the implant support system using the distal interface strut and implanted in the spine for some embodiments;

FIG. 3F depicts a perspective view of a cannulated guide for some embodiments;

FIG. 4 depicts an exemplary method for inserting an implant support system into a patient.

The drawing figures do not limit the present disclosure to the specific embodiments disclosed and described herein. The drawings are not necessarily to scale; emphasis is instead placed upon clearly illustrating the principles of the present disclosure.

### DETAILED DESCRIPTION

The following description of embodiments of the present disclosure references the accompanying illustrations that illustrate specific embodiments in which the present disclosure can be practiced. The embodiments are intended to describe aspects of the invention in sufficient detail to enable those skilled in the art to practice the present disclosure. Other embodiments can be utilized, and changes can be made without departing from the scope of the present disclosure. Therefore, the following detailed description is not to be taken in a limiting sense.

In this description, references to “one embodiment,” “an embodiment,” “embodiments,” “various embodiments,” “certain embodiments,” “some embodiments,” or “other embodiments” mean that the feature or features being referred to are included in at least one embodiment of the technology. Separate references to “one embodiment,” “an embodiment,” “embodiments,” “various embodiments,” “certain embodiments,” “some embodiments,” or “other embodiments” in this description do not necessarily refer to the same embodiment and are also not mutually exclusive unless so stated and/or except as will be readily apparent to those skilled in the art from the description. For example, a feature, structure, act, etc., described in one embodiment may also be included in other embodiments but is not necessarily included. Thus, the current technology can include a variety of combinations and/or integrations of the embodiments described herein.

Generally, embodiments of the current disclosure are directed toward stabilizing or otherwise supporting a spinal implant via a load-bearing support structure such as a strut implant. In this way, the strut implant may improve mechanical properties of a spinal fixation to reduce spinal implant deformity and breakage, such that fixating multiple un-injured vertebrae is unnecessary. Additionally, the strut implant may bolster mechanical properties of spinal fixation to reduce spinal implant deformity (e.g., a “toe in” of screws resulting in kyphosis) or breakage. Embodiments of the current disclosure are also directed to systems and methods for implementing an implant support system comprising a spinal implant and a strut implant providing load-bearing support to the spinal implant.

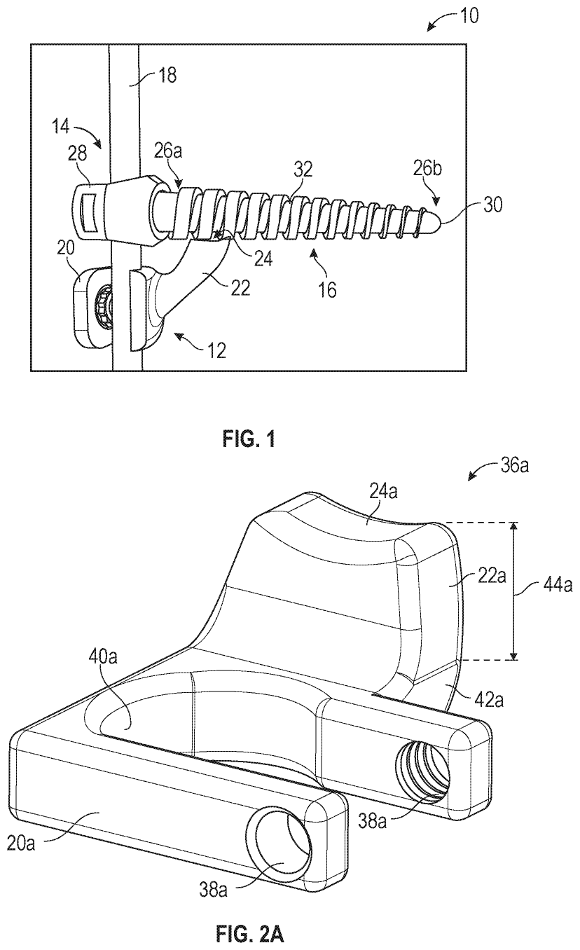

FIG. 1 depicts a perspective view of an exemplary implant support system 10 for some embodiments. The implant support system 10 may include a strut implant 12 (otherwise referred to herein as strut) and a spinal implant 14. The spinal implant 14 may provide fixation to the spine. The strut implant 12 may stabilize and support the spinal implant 14 by contacting a portion of the spinal implant 14 to share or offload forces acting on the spinal implant 14 (as shown in FIG. 1). In some embodiments, the strut implant 12 may contact a vertebra implanted with a portion of the spinal implant 14 to provide structural support, as discussed later.

The spinal implant 14 may be a pedicle screw-rod construct used to address spinal deformities and misalignments using segment fixation. For example, the spinal implant 14 may include a screw 16 (also referred to herein as pedicle screw) coupled to a rod 18, as shown in FIG. 1. The strut implant 12 may couple to the rod 18 and contact the screw 16 to thereby provide structural support to the spinal implant 14. This structural support may keep the spinal implant 14 from deforming, such as the screw 16 from breaking. Such structural support may improve mechanical properties of the spinal implant 14. For example, the support provided by strut implant 12 may increase the stability and/or rigidity of screw 16 in spinal implant 14 to improve mechanical properties of the spinal implant 14.

In some embodiments, as shown in FIG. 1, the strut implant 12 may be positioned underneath a portion of the spinal implant 14 and contact a bottom portion of the spinal implant 14 to share or offload compressive forces acting on the spinal implant 14. In some embodiments, the strut implant 12 may couple to the rod 18 at a position inferior to the screw 16 of the spinal implant 14 such that strut implant 12 contacts an underside of screw 16 to thereby support the spinal implant 14.

In some embodiments, strut implant 12 may be positioned above screw 16 and couple to rod 18 above screw 16 such that strut implant 12 contacts a top of screw 16 to thereby support the spinal implant 14. This may be particularly useful in providing support to rod-screw constructs with polyaxial screws that may toe-in to each other and accordingly cause kyphosis.

The strut implant 12 may couple to the spinal implant 14 via a base 20 and contact the spinal implant 14 via a strut extension 22. A support surface 24 of the strut extension 22 may contact a portion of the spinal implant 14 to provide structural support. For example, in some embodiments, the strut extension 22 defines a support surface 24. In some embodiments, when base 20 attaches to the spinal implant 14, the support surface 24 may contact a screw 16 of the spinal implant 14 to thereby provide structural support to the spinal implant 14, as shown in FIG. 1. The base 20 may couple to rod 18 of the spinal implant 14. In some embodiments, the strut extension 22 may extend from base 20.

The support surface 24 may contact the screw 16 of the spinal implant 14 within the implant support system 10, as shown in FIG. 1. In some embodiments, the support surface 24 of the strut implant 12 may contact a portion of a vertebra when inserted at the spine to provide structural support to the spinal implant 14 implanted in the vertebra. For example, the strut implant 12 may couple to rod 18 of spinal implant 14 and contact a vertebra implanted with screw 16 to thereby support the stability of the spinal implant 14.

As is well understood within the art, the pedicle screw-rod construct is a spinal implant utilized for segment fixation in which screws 16 act as anchors within the vertebra by threading into the vertebra. The screws 16 are coupled to rods 18 at a proximal end 26a of screws, as shown in FIG. 1, to support the screws 16 and form a rigid construct to restrict spinal movement to promote spinal fixation. A rod connector 28 couples the screw 16 to rod 18. The rod connector 28 may be a tulip connector or any other type of connector. The screw 16 includes a tip 30 at a distal end 26b that may be sharpened to enable drilling and tapping into bone.

In some embodiments, the screw 16 is monoaxial, and the monolithic structural nature of the monoaxial screw may include a junction between a screw head (not shown) and a screw shaft 32 at which stress concentrates and may cause mechanical failure of the screw 16, which causes spinal implant 14 breakage, requiring surgical intervention to strengthen the existing spinal implant 14 and replace the failed screw. In some embodiments, screw 16 is polyaxial and has a ball-shaped screw head (not shown) housed in the rod connector 28 that allows for movement of the screw 16 about the ball joint. Screw 16, when polyaxial, may experience slippage from their desired alignment (often referred to as “toeing-in” of screws). Such a mechanism of failure may contribute to or further perpetuate spinal deformity and alignment lost, such as spinal kyphosis, interbody subsidence, or a general deterioration in sagittal balance. Moreover, the slippage may lead to sciatica symptoms as nerve roots become pinched or other segment diseases resulting in immobility or difficultly in daily functioning that requires surgical intervention. To achieve greater stability and solve these known mechanical failures, existing fixation devices (such as the spinal implant 14) may use strut implant 12 to improve stability of spinal implant 14, thereby forming the implant support system 10.

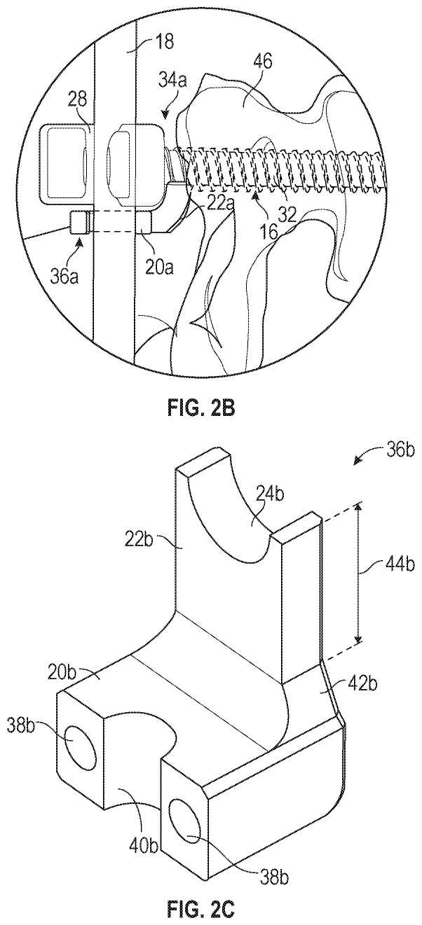

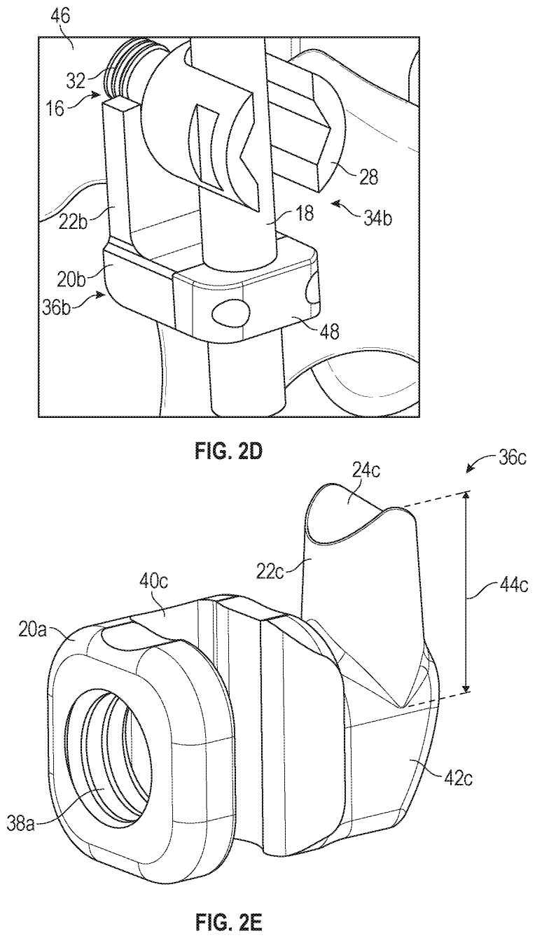

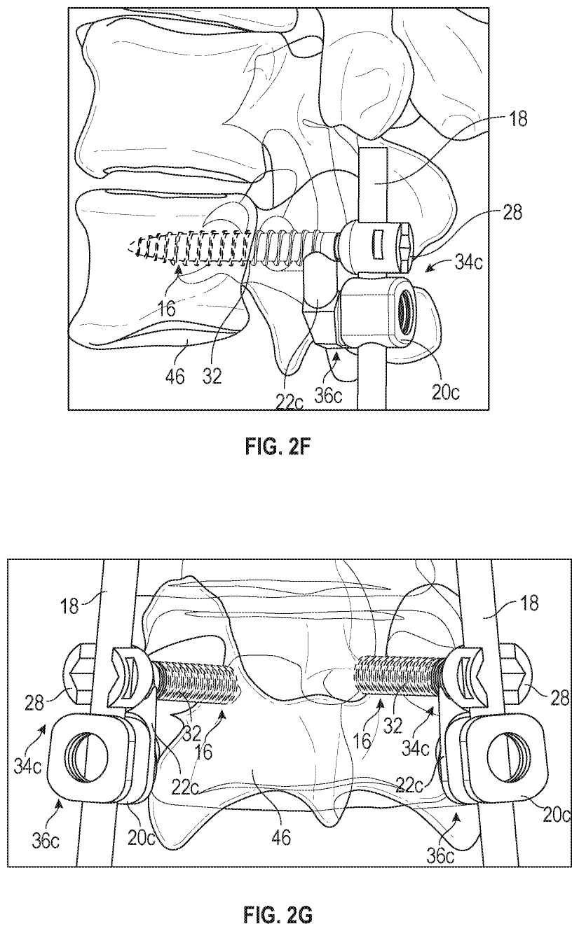

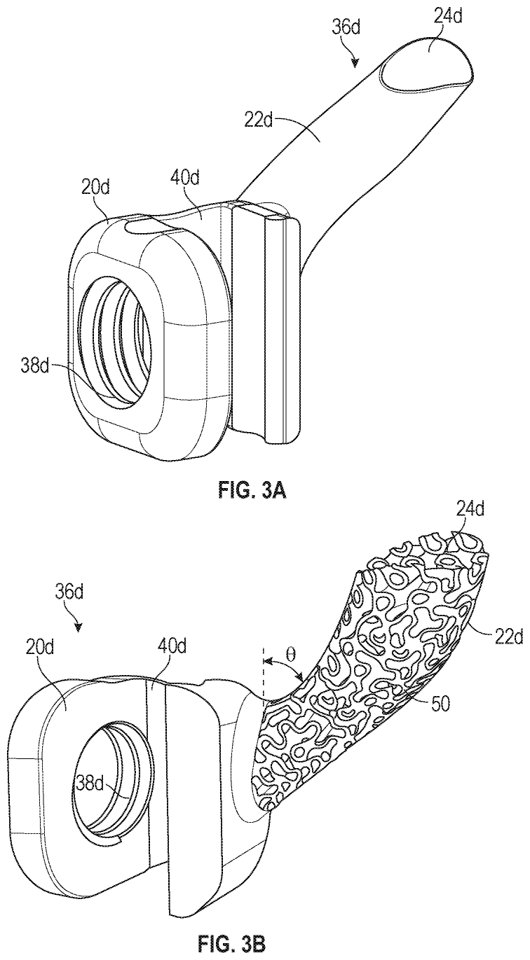

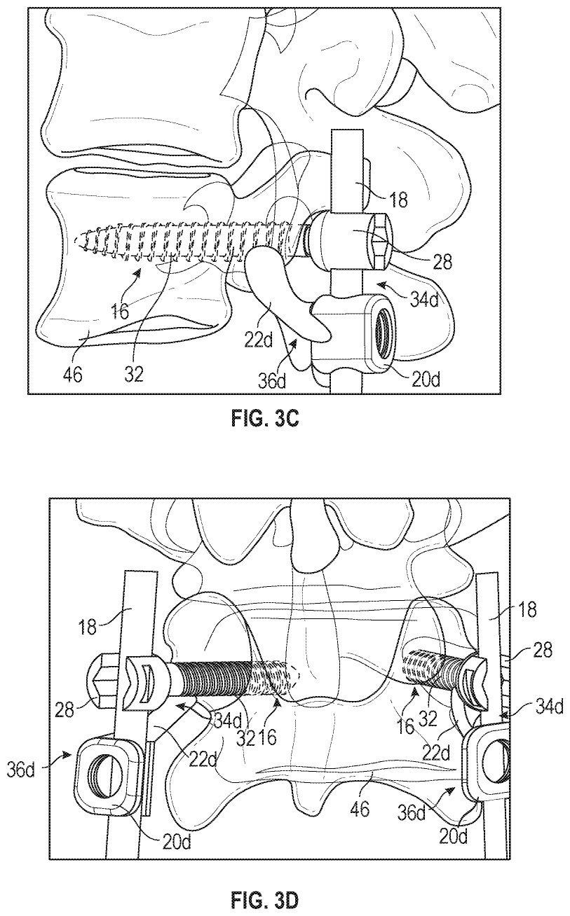

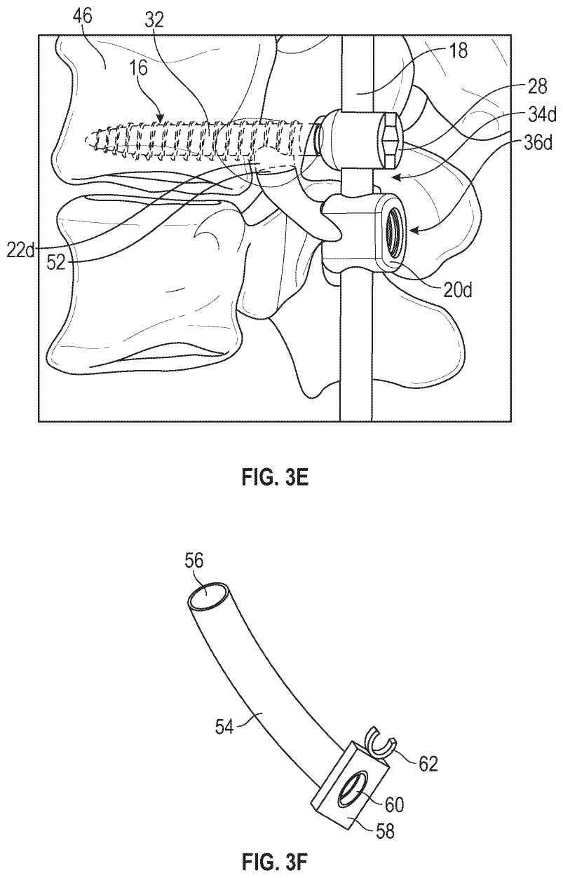

The strut implant 12 (and accordingly, implant support system 10 containing the strut implant 12) may be implemented in many forms (for instance, as the embodiments of implant support system 34a, 34b, 34c, and 34d correlating to FIGS. 2B, 2D, 2F-2G, and 3C-3E as indicated). For example, strut implant 12 may be a proximal interface strut 36a, 36b, 36c (as discussed with reference to FIGS. 2A-2G) or a distal interface strut 36d (as discussed with reference to 3A-3E). Both proximal interface strut 36a, 36b, 36c and distal interface strut 36d are each a strut implant 12. The proximal interface strut 36a, 36b, 36c (as discussed with reference to FIGS. 2A-2G) may provide contact support to spinal implant 14 by contacting spinal implant 14 outside of the screw/bone interface of the vertebra implanted with the spinal implant 14. The distal interface strut 36d (as discussed with reference to FIGS. 3A-3E) may provide contact support to spinal implant 14 by contacting the vertebra or contacting spinal implant 14 by passing through a portion of the vertebra implanted with the spinal implant 14.

Strut implant 12 improves the stability of spinal implant 14 and may mitigate the extent of segment fixation required for a spinal intervention. For example, less vertebrae may need to be fixated via the pedicle-screw rod construct when applying a strut implant 12, thereby decreasing the complexity and invasive of the surgical procedure and maximizing spinal movement. The strut implant 12 provides contact support (either to the vertebra or to the spinal implant 14 itself) to increase the strength of a pedicle screw-rod construct, thereby reducing the maximum stress. As such, the longevity of the implant construct may be extended, and implant failure may be prevented while patient mobility is not limited. As shown in FIG. 1, strut implant 12 may contact spinal implant 14 at shaft 32 of screw 16 to thereby provide contact support.

FIGS. 2A-2G depict a plurality of embodiments of a proximal interface strut 36a, 36b, 36c that each form a strut implant 12. FIG. 2A depicts a perspective view of an exemplary proximal interface strut 36a for some embodiments. FIG. 2B depicts a side view of an exemplary implant support system 34a for some embodiments using the proximal interface strut 36a and implanted in the spine. Proximal interface strut 36a is a strut implant 12 that may provide contact support to spinal implant 14 outside of the screw/bone interface of a vertebra, as shown in FIG. 2B. In some embodiments, proximal interface strut 36a may contact shaft 32 of screw 16, as shown in FIG. 2B. In some embodiments, proximal interface struts 36a, 36b, 36c may provide support to screw 16 outside the cortex of the vertebra and in front of the rod connector 28. The proximal interface strut 36a, 36b, 36c may improve the mechanical strength of a screw 16 that is polyaxial to be comparative to the mechanical strength of a screw 16 that is monoaxial.

The proximal interface struts 36a, 36b, 36c may provide additional support to the outer dense layer of cortical bone surrounding the screw 16 in a vertebra, also referred to as the cortex, by bolstering the shaft 32 inside the cortical bone. Notably, such support does not require any direct contact with the cortex itself and can achieve a desired improvement to rigidity via the interface between the strut implant 12 and the shaft 32. As such, proximal interface struts 36a, 36b, 36c may increase stability of any implant construct by dispersing stress but may be particularly relevant to stabilizing monoaxial and polyaxial screws directly at the rod-screw junction near the proximal end 26a of screw 16 of spinal implant 14.

In some embodiments, the proximal interface strut 36a, 36b, 36c may attach to the spinal implant 14 (e.g., at the rod 18, rod connector 28, or screw 16) such that the proximal interface struct 36a, 36b, 36c is located inferior to the bone/screw interface. For example, in some embodiments, the proximal interface strut 36a, 36b, 36c may be located inferior to screw 16. As such, the proximal interface strut 36a, 36b, 36c may extend vertically upward to contact the screw 16 and prevent downward toe-in of the screw 16. In some embodiments, the proximal interface strut 36a, 36b, 36c may couple to the spinal implant 14 such that the proximal interface strut 36a, 36b, 36c is located superior to the bone/screw interface. For example, in some embodiments, the proximal interface strut 36a, 36b, 36c may be located superior to screw 16. In such instances, the proximal interface strut 36a, 36b, 36c may extend vertically downward to contact the screw 16 and prevent upward toe-in of the screw 16.

Turning first to FIG. 2A, the proximal interface strut 36a includes a support surface 24a for contacting the screw 16 of spinal implant 14. The support surface 24a may extend from a base 20a of proximal interface strut 36a via strut extension 22a. The base 20a may secure the proximal interface strut 36a to spinal implant 14. In some embodiments, the base 20a may be secured to the rod 18, the screw 16, or the rod connector 28 of the spinal implant 14. In some embodiments, base 20a may couple to rod connector 28, such as to a tulip connector connecting screw 16 to rod 18. In some embodiments, the base 20a may attach laterally to rod 18, as shown in FIG. 2B. Attaching base 20a to a side of rod 18 may decrease the overall profile of the proximal interface strut 36a to make it easier to fit onto the spinal implant 14. In some embodiments, when base 20a attaches laterally to a side of rod 18, the support surface 24a is directed upward such that screw 16 may rest on support surface 24a.

In some embodiments, base 20a attaches to rod 18 via fastener. In some embodiments, base 20a may be a tulip connector and connect to rod 18 via the tulip connector. For instance, base 20a may define holes 38a for receiving a fastener therein to secure proximal interface strut 36a to spinal implant 14. In some embodiments, base 20a may attach to rod 18 via shims that surround the rod 18 such that tightening the shims together using a screw affixes an inner surface 40a of base 20a to the rod 18. In some embodiments, base 20a may attach to rod 18 via interference, may snap onto rod 18, or otherwise be secured to rod 18 of spinal implant 14. In some embodiments, base 20a may attach to spinal implant 14 via an attachable clamp, a hinged clamp, a set screw and grooved insert, or a partial collar.

In some embodiments, the base 20a may be secured to a portion of the screw 16 such as the screw head. In some embodiments, the base 20a may be c-shaped such that base 20a attaches to a side of rod 18 when secured to spinal implant 14. In some embodiments, base 20a may be U-shaped and attach to a rear of the rod 18.

The proximal interface strut 36a may include a neck 42a adjoined to or otherwise extending from the base 20a that connects strut extension 22a to base 20a. The strut extension 22a may be configured in a variety of geometries allowing access to the shaft 32 of screw 16 including, but not limited to, a rectangular prism or trapezoidal feature shown at FIG. 2A. In some embodiments, the strut extension 22a may be a three-dimensional flat construct. Moreover, the strut extension 22a may be monolithic or could couple to the neck 42a via a polyaxial joint similar to the previously described polyaxial screw.

Further, the strut extension 22a may have a length (L₁) 44a defined from the neck 42a to the support surface 24a at which the strut extension 22a may be in contact with the spinal implant 14. In some embodiments, the strut extension 22a and, thus, the length 44a is vertical. In some embodiments, the strut extension 22a projects at an angle diagonally from neck 42a and base 20a. The length (L₁) 44a of strut extension 22a may be selected based on patient geometry to provide an optimal fit for contacting the screw 16 of spinal implant 14 from rod 18 attachment.

The strut extension 22a terminates at a support surface 24a, which is the surface at which the proximal interface strut 36a provides contact support to the spinal implant 14. For the proximal interface strut 36a, the support surface 24a interfaces with the screw 16. In some embodiments, the support surface 24a may be saddle shaped to interface with the curved profile of screw 16 of spinal implant 14. The saddle shape may correspond to the overall diameter of the screw shaft, providing a generally close fit. Further still, the support surface 24a may be designed to match both the inner and outer diameters of screw 16 threads on shaft 32. In some embodiments, the support surface 24a may have a deep curvature to support sides of the shaft 32 of screw 16, as well as support the top or bottom of the shaft 32.

As shown in FIG. 2B, the proximal interface strut 36a of the implant support system 34a contacts the shaft 32 of screw 16 of spinal implant 14 when screw 16 is inserted in a vertebra 46. Proximal interface strut 36a may contact screw 16 of spinal implant 14 proximate the proximal end 26a of screw 16, as shown in FIG. 2B. Proximal interface strut 36a may contact shaft 32 of screw 16 under screw 16. The implant support system 34a may include any of the components of the spinal implant 14 (e.g., rod 18, screw 16, rod connector 28) and includes the proximal interface strut 36a of FIG. 2A. As shown in FIG. 2B, the C-shaped base 20a may be secured about the rod 18 such that the strut extension 22a extends superiorly toward the shaft 32 of the screw 16. The support surface of the proximal interface strut 36a contacts the shaft 32 outside the bone/screw interface of the vertebra.

FIG. 2C depicts a perspective view of a second embodiment of a proximal interface strut 36b. Proximal interface strut 36b is a strut implant 12. Much like proximal interface strut 36a, proximal interface strut 36b provides contact support to the spinal implant outside the bone/screw interface of the vertebra. Proximal interface strut 36b of FIG. 2C is substantially similar to proximal interface strut 36a in FIG. 2A, including a base 20b, inner surface 40b, holes 38b, support surface 24b, neck 42b, and strut extension 22b having length L₂ 44b. However, proximal interface strut 36b attaches to rod 18 anteriorly, rather than proximal interface strut 36a attaching to rod 18 laterally.

The neck 42b extends from a top surface of the base 20b rather than a side surface of the base 20a (as shown at FIG. 2A), thereby increasing the length L₂ 44b between support surface 24b and neck 42b. This ability to offset base 20b away from screw 16 advantageously permits the screw 16 to sit more rigidly by lowering the center of gravity of the entirety of the implant support system 34b, which may be particularly advantageous for spinal implants 14 having a polyaxial screw.

FIG. 2D depicts a perspective detailed view of an exemplary implant support system 34b for some embodiments using the second embodiment of the proximal interface strut 36b and implanted in the spine. As shown in FIG. 2D, the base 20b attaches lower on rod 18 away from screw 16 than base 20a of proximal interface strut 36a (as shown in FIG. 2B), thereby lowering the center of gravity and providing a longer strut extension 22b to provide increased stability that may be useful when applying to a spinal implant 14 using polyaxial screws. Otherwise, the proximal interface strut 36b supports the spinal implant 14 when inserted in vertebra 46 in substantially the same way as proximal interface strut 36a shown in FIG. 2B, as support surface 24b on strut extension 22b similarly contacts shaft 32 of screw 16 proximate the proximal end 26a of screw 16 and posterior to vertebra 46. Similarly, proximal interface strut 36b also contacts shaft 32 of screw 16 under screw 16.

As shown in FIG. 2D, the base 20b of proximal interface strut 36b may attach to rod 18 from the front of rod 18. Base 20b may attach to rod 18 via a cap 48. For example, in some embodiments, cap 48 may be fastened onto base 20b from the rear of rod 18. Cap 48 may define holes that coincide with holes 38b of base 20b such that the holes may receive fasteners to secure cap 48 to base 20b and thereby secure rod 18 between cap 48 and base 20b. In some embodiment, base 20b of proximal interface strut 36b may attach and be secured to rod 18 using substantially the same fastening mechanisms as described above for proximal interface strut 36a, such as through interference fit, shim tightening, clamps, a set screw and grooved insert, or a partial collar.

FIG. 2E depicts a perspective view of a third embodiment of a proximal interface strut 36c. Proximal interface strut 36c is a strut implant 12, and provides contact support to spinal implant 14 by contacting screw 16, much like proximal interface strut 36a, 36b. Proximal interface strut 36c of FIG. 2E is substantially similar to proximal interface strut 36a in FIG. 2A and proximal interface strut 36b in FIG. 2C, including a base 20c, inner surface 40c, hole 38c, support surface 24c, neck 42c, and strut extension 22c having length L₃ 44c. Similarly to FIG. 2A, the neck 42c extends from a side surface of the base 20c, as shown in FIG. 2E. The strut extension 22c is substantially similar to strut extension 22a and strut extension 22b, extending superior to neck 42c. In some embodiments, the strut extension 22c of proximal interface strut 36c may be cylindrical or rounded. Base 20c defines a hole 38c for receiving a fastener to secure proximal interface strut 36c to rod 18 of spinal implant 14, which is substantially similar to base 20a and base 20b.

FIG. 2F depicts a perspective view of a third embodiment of an implant support system 34c for some embodiments using the third embodiment of the proximal interface strut 36c and implanted in the spine. The proximal interface strut 36c provides support to spinal implant 14 in substantially the same way as proximal interface strut 36a, 36b, as the support surface 24c of strut extension 22c contacts screw 16 outside of the screw/bone interface of the vertebra 46. The base 20c may be secured to rod 18 by screwing a fastener into hole 38c and tightening the fastener against rod 18. The implant support system 34c may be substantially similar to the implant support system 34a, 34b of FIGS. 2B and 2D, including spinal implant 14 and its components (e.g., rod 18, screw 16, rod connector 28) and a proximal interface strut 36a, 36b, 36c.

FIG. 2G depicts a perspective view of multiple implant support systems 34c using the third embodiment of the proximal interface strut 36c implanted in the spine. In some embodiments, implant support systems 34c may be implemented with screw-rod constructs at a left and right lateral portion of a vertebra across the median sagittal plane of a patient. Multiple implant support systems 34c may use any of the proximal interface struts 36a, 36b, 36c to provide support to multiple rod-screw constructs across a vertebra. In some embodiments, the left and right lateral implementation may involve the inclusion of two rods 18, each dedicated to a lateral side as is standard in spinal fixation via rod-screw segment fixation. Thus, strut implants 12, such as proximal interface struts 36a, 36b, 36c may be used for each rod-screw interface to improve rigidity and stability of the spinal implant 14 within the implant support system 10.

FIGS. 3A-3E depict exemplary embodiments of a distal interface strut 36d that each form a strut implant 12 for providing support to the spinal implant 14. Distal interface struts 36d provide support to the spinal implant 14 on or through the vertebra, while proximal interface struts 36a, 36b, 36c provide support to the spinal implant 14 outside of the vertebra (e.g., supporting shaft 32 of screw 16 outside the screw/bone interface of the vertebra). The distal interface struts 36d may be elongated when compared to the proximal interface struts 36a, 36b, 36c, since the distal interface struts 36d provide contact support via support surface 24d contacting screw 16 at a location more proximate to distal end 26b of screw 16 than proximal interface struts 36a, 36b, 36c.

In some embodiments, the distal interface strut 36d provides an increase in rigidity to the spinal implant 14 via contact support of shaft 32 of screw 16 occurring through a cortical window within the vertebra. In some embodiments, the distal interface strut 36d provides support directly to the cortical bone of the vertebra itself to provide structural support to the spinal implant 14.

Accordingly, distal interface struts 36d provide additional support to the cortex and/or screw 16 by shifting the location of contact for the support surface 24d distally along the shaft 32. Such a shift further stabilizes spinal implant 14 by dispersing stress to a location distally offset from the base 20d of the strut implant 12 and, accordingly, dispersing stress otherwise concentrated at the rod/screw interface. Additionally, distal interface struts 36d may be longer than proximal interface struts 36a, 36b, 36c, allowing further dispersing of stress otherwise concentrated at the rod/screw interface by base 20d being longitudinally offset from the rod/screw connection. Therefore, incorporation of a distal interface strut 36d improves load distribution and allows for support extending further anteriorly relative to a vertebra.

FIG. 3A depicts a perspective view of an exemplary embodiment of a distal interface strut 36d. Distal interface strut 36d forms the strut implant 12 shown in FIG. 1. As shown, distal interface strut 36d may be substantially similar to the proximal interface struts 36a, 36b, 36c, including a base 20d, inner surface 40d, defining a hole 38d, support surface 24d, and having a strut extension 22d. In some embodiments, the strut extension 22d extends from a side surface of the base 20d. In some embodiments, the strut extension 22d may extend directly from a side surface of base 20d without extending from a neck 42d, as shown in FIG. 3A. The strut extension 22d may curve outward from base 20d, rather than vertically extend such as strut extensions 22a, 22b, and 22c of proximal interface struts 36a, 36b, 36c. The strut extension curving outward allows the support surface 24d to access distal contact points along screw 16 or on a vertebra in the distal interface strut 36d. In some embodiments, strut extension 22d may curve diagonally outward to extend toward screw 16 of spinal implant 14 to reach distally located contact points, when compared to proximal interface struts 36a, 36b, 36c.

In some embodiments, the distal interface strut 36d may attach to the spinal implant, including at the rod 18, rod connector 28, or screw 16, via the base 20 such that the distal interface strut 36d is located inferior to (i.e., below) the bone/screw interface. For example, in some embodiments, the distal interface strut 36d may be located inferior to the screw 16. As such, the distal interface strut 36d may extend vertically upward to contact the screw 16 or the cortical bone and prevent downward toe-in of the screw 16. In some embodiments, the distal interface strut 36d may be secured to the spinal implant 14 such that the distal interface struct is located superiorly to (i.e., above) the bone/screw interface. For example, in some embodiments, the distal interface strut 36d may be located superior to the screw 16. In such instances, the distal interface strut 36d may extend vertically downward to contact the screw 16 or the cortical bone and prevent upward toe-in of the screw 16.

In some embodiments, the strut extension 22d may extend at an angle Θ (as shown in FIG. 3B) relative to the side surface of the base 20d. The range of angle Θ may depend on the vertebra or other bony structure impacting the interfacing of the distal interface strut 36d. In some embodiments, the angle Θ may be within a range of between 0 and 30 degrees; however, it will be appreciated that the angle Θ may be any angle permitting access to support the cortical bone (or support screw 16 through cortical window).

Further, the strut extension 22d and the support surface 24d may feature curvature that allows greater contact with the screw 16 within the more confined spaces of the vertebra. The support surface 24d is substantially the same as support surface 24a, 24b, 24c in the distal interface struts 36d located on strut extension 22d. In some embodiments, the support surface 24d may have a deep curvature to support sides of the shaft 32 of screw 16, as well as support the top or bottom of the shaft 32.

FIG. 3B depicts a perspective view of the distal interface strut 36d containing a lattice structure 50 for some embodiments. The lattice structure 50 is highly porous and may deform to have a more congruent support surface 24d for mating with shaft 32 of screw 16 or mating with cortical bone on a vertebra. In some embodiments, the support surface 24d comprises a lattice structure 50. In some embodiments, part of the strut extension 22d or all of the strut extension 22d, including the support surface 24d, may contain a lattice structure 50 (as depicted in FIG. 3B). Lattice structure 50 may allow for bone in-growth over time to create more biologic distal interface struts 36d that may improve the speed and quality of fixation. In some embodiments, the lattice structure 50 may be packed with bone graft materials, such as osteo-conductive, osteo-inductive, and osteogenic grafts to further encourage bony fusion and in-growth.

It will be appreciated that, while largely applicable for distal interface strut 36d embodiments due to their close proximity to vertebral bodies, cortical bones, and other bony structures, the lattice structure 50 may be utilized for any of the proximal interface struts 36a, 36b, 236c, particularly for the strut extension 22a, 22b, 22c and the support surface 24a, 24b, 24c.

FIG. 3C depicts a perspective detailed view of an exemplary implant support system 34d using the distal interface strut 36d and implanted in the spine. The implant support system 34d of FIG. 3C is the same implant support system 34 shown in FIG. 1. As shown in FIG. 3C, the strut extension 22d may contact cortical bone of the vertebra 46 to provide structural support to the spinal implant 14. The strut extension 22d contacts the cortical bone via support surface 24d. The support surface 24d may support the screw 16 without contacting the shaft 32. In some embodiments, the support surface 24d may contact the cortical bone on any cortical surface including, but not limited to, a cortical surface that does not pass a transverse process anteriorly, as depicted in FIG. 3C. The implant support system 34d, as shown in FIG. 3C, may include any of the components of the spinal implant 14 (e.g., rod 18, screw 16, rod connector 28), and includes the distal interface strut 36d of FIG. 3A.

FIG. 3D depicts a perspective detailed view of multiple implant support systems 34d each using the distal interface strut 36d for providing support to the cortex via contact of the support surface 24d on strut extension 22d with the cortical bone. The implant support systems 34d may be implemented, for instance, with screw-rod constructs at a left and right lateral portion of a vertebra across the median sagittal plane of a patient. Multiple implant support systems 34d may use any distal interface struts 36d to provide support to multiple rod-screw constructs across a vertebra 46, as shown by the two distal interface struts 36d in FIG. 3D. In some embodiments, the left and right lateral implementation may involve the inclusion of two rods 18, each dedicated to a lateral side as is standard in spinal fixation via rod-screw segment fixation. Each of the multiple implant support systems 34d include the previously described rod 18, screw 16, and rod connector 28. Further, the implant support systems 34d each comprise a strut implant 12 (in the form of distal interface struts 36d) contacting the bone cortex. As such, strut implants 12, such as distal interface struts 36d, may be utilized to provide support directly to the vertebra 46, and the support surface 24d may be in direct contact with the bony cortical surface itself at an external surface of the vertebra 46.

While not shown in the figures, it will be appreciated that multiple implant support systems 10 may be integrated onto a single rod 18 at various vertebral levels. For instance, a first strut implant 12 may support an inferior aspect of one screw 16 while a second strut implant 12 may support a superior aspect of another screw 16 in a spinal implant 14. Moreover, any combination of the described strut implants 12 may be implemented within the same patient at various vertebral levels. For example, both distal interface struts 36d and proximal interface struts 36a, 36b, 36c may be used in combination to provide support for a spinal implant 14 within an implant support system 10.

FIG. 3E depicts a perspective detailed view of the implant support system 34d using the distal interface strut 36d and implanted in the spine. As shown in FIG. 3E, the strut extension 22d contacts the shaft 32 of screw 16 within a vertebra 46 through a cortical window 52. In some embodiments, the strut extension 22d of distal interface strut 36d passes through a drilled hole in vertebra 46 to contact shaft 32 within vertebra 46. The support surface 24d of strut extension 22d may contact shaft 32 of screw 16 within vertebra 46. The implant support system 34d, as shown in FIG. 3E, may include any of the components of the spinal implant 14 (e.g., rod 18, screw 16, rod connector 28), and includes the distal interface strut 36d of FIG. 3A.

The strut extension 22d accesses the screw 16 via a cortical window 52 such that the support surface 24d contacts the shaft 32 despite access limitations resulting from the physical components of the vertebra or to avoid critical anatomy such as nearby nerve roots and vascular structures. The cortical window 52 may be a hole or partial hole created by removing a portion of the vertebra in order to allow for greater anterior extension of the strut implant 12, such as the distal interface strut 36d. The strut implant 12 implemented embodiments accessing the screw 16 through a cortical window 52 may have any portion of the strut extension 22d housed within the cortical window 52.

FIG. 3F depicts a perspective view of a cannulated guide 54 for some embodiments. The cannulated guide 54 is used to access cortical bone of a vertebra 46 for inserting the distal interface strut 36d for some embodiments. The cannulated guide 54 may be curved to provide adequate access to the cortical bone at a desired location of a vertebra 46, as is needed for embodiments in which the distal interface strut 36d contacts the screw 16 through a cortical window 52 (such as, for instance, that which is depicted in FIG. 3E). The cannulated guide 54 defines an aperture 56 extending therethrough. In some embodiments, the diameter of the aperture 56 is similar to the diameter of the support surface 24d and strut extension 22d of distal interface strut 36d.

The cannulated guide 54 may be inserted and advanced such that one end of the cannulated guide 54 rests against a surface of a vertebra to define the location of the cortical window 52. The other end of the cannulated guide 54 comprises a cannula connector 58 defining a hole 60 therethrough aligning with the aperture 56. The cannula connector 58 may include a coupler 62 extending from at least one surface to couple the cannulated guide to the spinal implant 14. In some embodiments, the coupler 62 may adjoin the cannulated guide 54 directly to the rod 18. In some embodiments, the coupler 62 may attach the cannulated guide 54 to the screw 16 or the rod connector 28. The coupler 62 may be a clamp, clip, or any other coupler for coupling the cannulated guide 54 to the spinal implant 14. Attachment of the cannulated guide 54 to the spinal implant 14 creates an accurate guide path for strut extension 22d of distal interface strut 36d.

A drill may be inserted through the hole 60 of the cannula connector 58 and the aperture 56 to drill a hole forming the cortical window 52. The drill (not shown) may be a flexible drill. The drilled hole may correspond with or closely match the outer diameter of the strut extension 22d, so the strut extension 22d may be inserted through the cortical window 52 without compromising stability by making a large drill hole.

The strut implant 12 (e.g., proximal surface strut 36a,36b, 36c, or distal surface strut 36d) may consist of biocompatible, medical-grade materials such as, but not limited to, titanium (e.g., commercially pure titanium, Grade 2 titanium, Ti-6Al-4V, Ti-6Al-4V ELI), stainless steel (e.g., 316L, 316LVM), carbon fiber, corrosion resistant alloys (e.g., nitinol, tantalum, cobalt-chrome, titanium alloy), or other composite materials or polymers, cobalt chrome. The strut implants 12 can be manufactured using a variety of techniques including injection molding, CNC, additive manufacturing, and casting. However, it will be appreciated that the support surface 24 and/or the strut extension 22 may be an alternative material than the rest of the strut implant 12. For instance, the support surface 24 may be made of compliant or deformable material to allow for the support surface 24 to mold to the shaft 32 of the screw 16. Such deformable surface may be made of a softer material than the base 20 and strut extension 22 of the strut including but not limited to polymers and lattice structures.

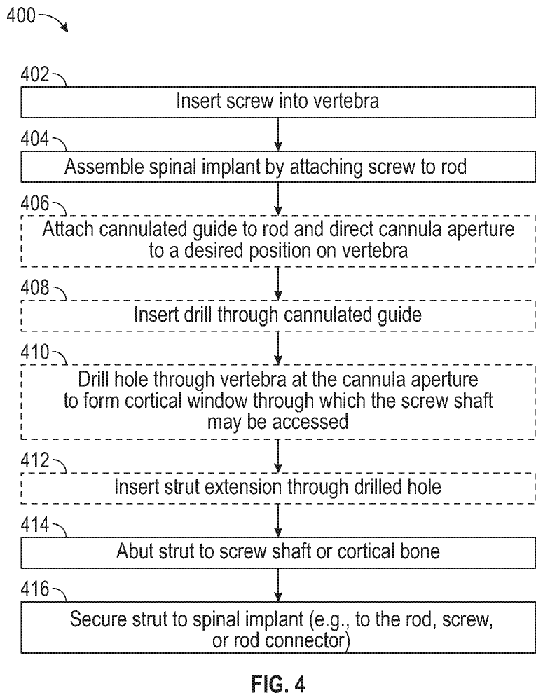

Turning now to FIG. 4, FIG. 4 depicts an exemplary method 400 for inserting an implant support system 10 into a patient in accordance with embodiments of the present disclosure. Exemplary method 400 may be performed with the implant support systems 10 and, thus, the strut implants 12 (including proximal interface struts 36a, 36b, 36c and distal interface struts 36d shown in FIGS. 2A-2G, and 3A-3E), as well as the spinal implant 14; however, other system configurations may also implement the method.

At step 402, the screw 16 may be inserted into the posterior lumbar of a patient. For instance, the screw 16 may be inserted into the vertebra 46 at the vertebral level of interest. In some embodiments, this may include two vertebral levels, one of which is above a vertebra requiring stabilization. In some embodiments, a screw 16 may be inserted into two vertebral levels such as an anterior and a superior vertebral level relative to a spinal fracture or deformity.

At step 404, the spinal implant 14 may be assembled. That is, the screw 16 may be attached to the rod 18. In some embodiments, the screw 16 may be secured to the rod 18 via the rod connector 28, which may be, e.g., a tulip connector.

At optional step 406, a cannulated guide 54 may be utilized to establish or further define a cortical bone window for embodiments in which a distal interface strut 36d has a support surface 24d contacting the screw 16 within vertebra 46. The cannulated guide 54 may be advanced to an area of interest of the vertebra through which the strut implant 12 may support the spinal implant 14. As such, the specific location of the cortical window 52 may be defined by the placement of the aperture 56 of cannulated guide 54.

At optional step 408, a flexible drill may be inserted through the cannulated guide 54 and directed toward the portion of the vertebra previously defined by the aperture 56 at step 406.

At optional step 410, a hole may be optionally drilled through the vertebra at the portion defined by the aperture 56 to form a cortical window 52. Drilling of the hole establishes the cortical window 52 through which the shaft 32 of screw 16 within vertebra 46 may be accessed. This step is relevant for embodiments involving contact support through a cortical window 52, such as when using a distal interface strut 36d.

At optional step 412, the strut extension 22d may be inserted through the drilled hole. As the diameter of the aperture 56 and, thus, of the drilled hole, corresponds with the footprint of the support surface 24d, the strut extension 22d may be inserted without a substantial clearance between the strut implant 12 and the drilled hole such that stabilization is not impacted.

At step 414, the strut implant 12 may be placed in contact with the spinal implant 14, abutting the support surface 24 to the shaft 32 of screw 16 or the cortical bone of vertebra 46. For example, the distal interface strut 36d may be placed in contact with the spinal implant 14 via placing the support surface 24d of strut extension 22d on shaft 32 of screw 16 within vertebra 46 through the cortical window 52 or placing the support surface 24d on cortical bone along outer surface of vertebra 46. The proximal interface strut 36a, 36b, 36c may be placed in contact with the spinal implant 14 via placing the support surface 24a, 24b, 24c on shaft 32 of screw outside the screw/bone interface of the vertebra 46.

At step 416, the strut implant 12 may be secured to the spinal implant 14. For instance, strut implant 12 may be affixed to the rod 18, rod connector 28, or screw 16 by fastening the base 20a, 20b, 20c, 20d to such feature. As described, multiple implant support systems 10 may be integrated onto a single rod 18 at various vertebral levels to provide support over several vertebrae. For instance, a first strut implant 12 may support a first screw 16 in a first vertebra, while a second strut implant 12 supports a second screw 16 in a second vertebra, to provide maximum support across an entire pedicle screw-rod segment construct, such as across vertebral joints that are fixated. The multiple implant support system 10 may be secured at step 416 by affixing the strut implant 12 to the rod 18 or rod connector 28.

Clause 1. A strut implant comprising: a base configured to couple to a spinal implant; a strut extension extending from the base, wherein the strut extension defines a support surface, wherein the support surface is configured to support a screw of the spinal implant or cortical bone implanted with the spinal implant to provide structural support.

Clause 2. The strut implant of clause 1, wherein the support surface is saddle-shaped.

Clause 3. The strut implant of clause 1 or clause 2, wherein the strut extension extends vertically relative to the base.

Clause 4. The strut implant of any of clause 1-3, wherein the strut extension extends diagonally outward from the base.

Clause 5. The strut implant of any of clause 1-4, further comprising: a neck attached to the base, wherein the strut extension extends vertically from the neck.

Clause 6. The strut implant of any of clause 1-5, wherein the strut extension is curved.

Clause 7. An implant support system, comprising: a spinal implant for providing spinal fixation, comprising: at least one screw; and at least one rod coupled to the at least one screw, wherein the at least one screw is configured to be inserted into a vertebra; and at least one strut implant, comprising: a base coupling the at least one strut implant to the spinal implant; and a strut extension extending from the base and defining a support surface, wherein the support surface is configured to contact the at least one screw to thereby provide structural support to the spinal implant.

Clause 8. The implant support system of clause 7, wherein the strut extension is configured to redistribute a stress concentrated at the at least one screw and/or the at least one rod to provide improved mechanical stability and thereby prevent mechanical failure of the spinal implant.

Clause 9. The implant support system of clause 7 or clause 8, wherein the spinal implant further comprises: a rod connector coupling the at least one screw to the at least one rod, wherein the base attaches to at least one of the at least one screw, the at least one rod, or the rod connector.

Clause 10. The implant support system of any of clauses 7-9, wherein the base defines a set of holes, and a fastener is received in the set of holes to couple the base to the at least one rod, thereby securing the at least one strut implant to the spinal implant.

Clause 11. The implant support system of any of clauses 7-10, wherein the at least one screw comprises a shaft, and the support surface contacts the shaft of the at least one screw.

Clause 12. The implant support system of any of clauses 7-11, wherein the at least one strut implant is a proximal interface strut implant, and the support surface is configured to contact the shaft of the at least one screw outside of a screw/bone interface when the implant support system is implanted at a spine.

Clause 13. The implant support system of any of clauses 7-12, wherein the at least one strut implant is a distal interface strut, and the support surface is configured to contact the shaft of the at least one screw located within the vertebra when the implant support system is implanted at a spine.

Clause 14. The implant support system of any of clauses 7-13, wherein the spinal implant is located superior to the at least one screw, wherein the strut extension extends vertically downward to contact the shaft via the support surface.

Clause 15. The implant support system of any of clauses 7-14, wherein the spinal implant is located inferior to the at least one screw, wherein the strut extension extends vertically upward to contact the shaft via the support surface

Clause 16. The implant support system of any of clauses 7-15, wherein at least one of strut extension or the support surface comprise a lattice structure.

Clause 17. The implant support system of any of clauses 7-16, wherein the distal interface strut extends diagonally outward from the base.

Clause 18. An implant support system, comprising: a spinal implant, comprising: a pedicle screw configured to screw into a vertebra, a rod, wherein the pedicle screw is coupled to the rod; and a strut implant, comprising: a base coupling the strut implant to at least one of the pedicle screw or the rod; and a strut extension extending from the base, wherein the strut extension forms a support surface, wherein the support surface is configured to abut cortical bone of the vertebra when the pedicle screw is screwed into the vertebra, wherein the support surface is configured to support the spinal implant to redistribute forces applied to the pedicle screw.

Clause 19. The implant support system of clause 18, wherein the strut extension comprises a lattice structure.

Clause 20. The implant support system of clause 18 or clause 19, wherein the support surface is formed from a lattice structure.

Clause 21. The implant support system of any of clauses 18-20, wherein the base is coupled to the rod underneath the pedicle screw, wherein the strut extension extends vertically upward such that the support surface contacts the cortical bone of the vertebra.

Clause 22. The implant support system of any of clauses 18-21, wherein the support surface is saddle-shaped.

Clause 23. The implant support system of any of clauses 18-22, wherein the base is coupled to the rod above the pedicle screw, wherein the strut extension extends vertically downward such that the support surface contacts the cortical bone of the vertebra.

Clause 24. The implant support system of any of clauses 18-23, wherein the lattice structure is configured to receive bony in-growth material to promote bony in-growth on the strut implant.

Clause 25. The implant support system of any of clauses 18-24, wherein the strut extension forms an angle relative to a side of the base.

Clause 26. A method for stabilizing a spinal implant, comprising: providing the spinal implant, the spinal implant comprising: at least one screw comprising a shaft; and at least one rod; providing at least one strut implant, the at least one strut implant comprising: a base; and an extension extending from the base, wherein the extension forms a support surface; inserting the at least one screw into a vertebra; attaching the at least one screw to the at least one rod; placing the support surface against the shaft of the at least one screw; and attaching the base of the at least one strut implant to the at least one rod or the at least one screw.

Clause 27. The method of clause 26, further comprising: prior to placing the at least one strut implant against the shaft, placing a cannulated guide against the vertebra; and drilling a hole in the vertebra to form a cortical window.

Clause 28. The method of clause 26 or clause 27, further comprising: placing the extension of the at least one strut implant through the cortical window until the support surface abuts the shaft of the at least one screw.

Although the present disclosure has been described with reference to the embodiments illustrated in the attached drawing figures, it is noted that equivalents may be employed, and substitutions made herein without departing from the scope of the present disclosure as recited in the claims.

Having thus described various embodiments of the present disclosure, what is claimed as new and desired to be protected by Letters Patent includes the following:

1. An implant support system, comprising:
a spinal implant for providing spinal fixation, comprising:
at least one screw; and
at least one rod coupled to the at least one screw,
wherein the at least one screw is configured to be inserted into a vertebra; and
at least one strut implant, comprising:
a base coupling the at least one strut implant to the spinal implant; and
a strut extension extending from the base and defining a support surface,
wherein the support surface is configured to contact the at least one screw to thereby provide structural support to the spinal implant.

2. The implant support system of claim 1, wherein the strut extension is configured to redistribute a stress concentrated at the at least one screw and/or the at least one rod to provide improved mechanical stability and thereby prevent mechanical failure of the spinal implant.

3. The implant support system of claim 1, wherein the spinal implant further comprises:
a rod connector coupling the at least one screw to the at least one rod,
wherein the base attaches to at least one of the at least one screw, the at least one rod, or the rod connector.

4. The implant support system of claim 1, wherein the at least one screw comprises a shaft, and the support surface contacts the shaft of the at least one screw.

5. The implant support system of claim 4, wherein the at least one strut implant is a proximal interface strut implant, and the support surface is configured to contact the shaft of the at least one screw outside of a screw/bone interface when the implant support system is implanted at a spine.

6. The implant support system of claim 4, wherein the at least one strut implant is a distal interface strut, and the support surface is configured to contact the shaft of the at least one screw located within the vertebra when the implant support system is implanted at a spine.

7. The implant support system of claim 4, wherein the strut implant is located superior to the at least one screw,
wherein the strut extension extends vertically downward to contact the shaft via the support surface.

8. The implant support system of claim 4, where in the strut implant is located inferior to the at least one screw,
wherein the strut extension extends vertically upward to contact the shaft via the support surface.

9. The implant support system of claim 1, wherein at least one of the strut extension or the support surface comprise a lattice structure.

10. The implant support system of claim 6, wherein the distal interface strut extends diagonally outward from the base.

11. An implant support system, comprising:
a spinal implant, comprising:
a pedicle screw configured to screw into a vertebra,
a rod,
wherein the pedicle screw is coupled to the rod; and
a strut implant, comprising:
a base coupling the strut implant to at least one of the pedicle screw or the rod; and
a strut extension extending from the base,
wherein the strut extension forms a support surface,
wherein the support surface is configured to abut cortical bone of the vertebra when the pedicle screw is screwed into the vertebra,
wherein the support surface is configured to support the spinal implant to redistribute forces applied to the pedicle screw and the rod.

12. The implant support system of claim 11, wherein the strut extension comprises a lattice structure.

13. The implant support system of claim 11, wherein the support surface is formed from a lattice structure.

14. The implant support system of claim 11, wherein the base is coupled to the rod underneath the pedicle screw,
wherein the strut extension extends vertically upward such that the support surface contacts the cortical bone of the vertebra.

15. The implant support system of claim 11, wherein the base is coupled to the rod above the pedicle screw,
wherein the strut extension extends vertically downward such that the support surface contacts the cortical bone of the vertebra.

16. The implant support system of claim 13, wherein the lattice structure is configured to receive bony in-growth material to promote bony in-growth on the strut implant.

17. The implant support system of claim 11, wherein the strut extension forms an angle relative to a side of the base.

18. A method for stabilizing a spinal implant, comprising:
providing the spinal implant, the spinal implant comprising:
at least one screw comprising a shaft; and
at least one rod;
providing at least one strut implant, the at least one strut implant comprising:
a base; and
an extension extending from the base,
wherein the extension forms a support surface;
inserting the at least one screw into a vertebra;
attaching the at least one screw to the at least one rod;
placing the support surface against the shaft of the at least one screw; and
attaching the base of the at least one strut implant to the at least one rod or the at least one screw.

19. The method of claim 18, further comprising:
prior to placing the at least one strut implant against the shaft, placing a cannulated guide against the vertebra; and
drilling a hole in the vertebra to form a cortical window.

20. The method of claim 19, further comprising:
placing the extension of the at least one strut implant through the cortical window until the support surface abuts the shaft of the at least one screw.
